(19) United States
(12) Patent Application Publication
(10) Pub. No.: US 2016/0019359 A1
(43) Pub. Date: Jan. 21, 2016

(54) SELECTING A DEVICE TO TREAT DISORDERED BREATHING

(71) Applicant: KONINKLIJKE PHILIPS N.V., Eindhoven (NL)

(72) Inventor: Eugene Nelson SCARBERRY, TRAFFORD, PA (US)

(21) Appl. No.: 14/772,213

(22) PCT Filed: Feb. 28, 2014
(86) PCT No.: PCT/IB2014/059332

§ 371 Date: Sep. 2, 2015

Related U.S. Application Data
(60) Provisional application No. 61/772,594, filed on Mar. 5, 2013.

(51) Int. Cl. G06F 19/00; A61M 16/06

(52) U.S. Cl. CPC G06F 19/3406; A61M 16/0666; A61M 2016/0661; A61M 2230/40

(57) ABSTRACT

A method for selecting a device adapted to treat disordered breathing in a patient with one or more comorbid diseases is provided. More particularly, a method for reducing a potential group of patient interface devices adapted to treat disordered breathing of a patient to an optimized group, includes: prescreening the patient; determining a measure of a comorbidity of the patient from the prescreening; comparing the measure of the comorbidity to a corresponding predetermined threshold; and determining the optimized group from the potential group. The optimized group may include a reduced number of devices from which a particular device is then selected or may include only a single particular device.

### CROSS-REFERENCE TO RELATED APPLICATIONS

This patent application claims the priority benefit under 35 U.S.C. §119(e) of U.S. Provisional Application No. 61/772,594 filed on Mar. 5, 2013, the contents of which are herein incorporated by reference.

### BACKGROUND OF THE INVENTION

1. Field of the Invention

The present invention pertains generally to the treatment of disordered breathing, and, more particularly, to a method for selecting a patient interface device or a component of a patient interface device to treat a patient who suffers from disordered breathing from amongst a plurality of known patient interface devices or components based on at least one comorbidity of the patient.

2. Description of the Related Art

It is well known that many individuals suffer from disordered breathing during sleep. Obstructive sleep apnea (OSA), which affects millions of people throughout the world, is a common example of such disordered breathing. OSA is a condition in which sleep is repeatedly interrupted by an inability to breathe. This inability to breathe is typically caused by intermittent obstruction of a person's airway. Obstruction of the airway is believed to be due, at least in part, to a general relaxation of the muscles which stabilize the upper airway segment. When these muscles relax, the surrounding tissues collapse thereby obstructing the airway.

Those afflicted with OSA experience sleep fragmentation and intermittent cessation of ventilation during sleep with potentially severe degrees of oxyhemoglobin desaturation. These symptoms may be translated clinically into extreme daytime sleepiness, cardiac arrhythmias, pulmonary-artery hypertension, congestive heart failure and/or cognitive dysfunction. Other consequences of OSA include right ventricular dysfunction, carbon dioxide retention during sleep, as well as wakefulness, and continuous reduced arterial oxygen tension. Sleep apnea sufferers may be at risk for excessive mortality from these factors as well as by an elevated risk for accidents while driving and/or operating potentially dangerous equipment.

A person may suffer from the adverse effects discussed above even where only partial obstruction of the airway occurs. Partial obstruction of the airway typically results in shallow breathing referred to as a hypopnea. Other types of disordered breathing include upper airway resistance syndrome (UARS) and vibration of the airway, such as vibration of the pharyngeal wall, commonly referred to as snoring. It is also known that snoring can accompany closure of the airway leading to UARS, hypopnea, or apnea. Thus, snoring serves as an indicator that the patient is experiencing abnormal breathing.

Milder cases of disordered breathing may be treated using an oral appliance such as a mandibular advancement device (MAD). A MAD is generally structured to pull a patient's lower jaw (mandible) forward relative to their upper jaw (maxilla), which in turn, opens the patient's airway.

More severe cases of disordered breathing may be treated by applying a continuous positive air pressure (CPAP) to the patient's airway. This positive pressure effectively “splints” the airway, thereby maintaining an open passage to the lungs. It is also known to provide a positive pressure therapy in which the pressure of gas delivered to the patient varies with the patient's breathing cycle, or varies with the patient's effort, to increase the comfort to the patient. One such pressure support technique is referred to as bi-level pressure support (e.g., BiPAP® bi-level pressure support), in which the inspiratory positive airway pressure (IPAP) delivered to the patient is higher than the expiratory positive airway pressure (EPAP) delivered to the patient. Other pressure support therapies and/or ventilation therapies are known and employed for the treatment of disordered breathing.

Pressure support and ventilation therapies may be non-invasively delivered to the patient. A patient interface device, generally placed on the face of a patient, facilitates the delivery of a flow of breathing gas from a pressure/flow generating device (e.g., a ventilator, pressure support device, etc.) to the airway of the patient. Patient interface devices include nasal prongs, nasal masks, full nasal/oral masks, and total face masks, among others. The pressure of the flow of breathing gas delivered to the patient is generally derived through a titration process administered by a sleep technician. The pressure at which the flow of breathing gas is delivered, herein referred to as the “therapeutic pressure”, is generally between 4 cm H₂O and 40 cm H₂O, although other pressures may be employed.

Patient comfort is maximized by providing the flow of breathing gas at a minimum effective therapeutic pressure (i.e., at the minimum pressure necessary to keep the patient's airway open). Furthermore, adverse conditions (for example and without limitation, mask leakage, skin breakdown caused by mask over-tightening, and acrophage) may be avoided at lower therapeutic pressures. Improved patient compliance with the prescribed therapy is achieved by increasing patient comfort and decreasing the likelihood that the patient experiences adverse conditions.

The particular device used to treat disordered breathing (e.g., a particular oral appliance or a particular patient interface device) is an important component for ensuring patient compliance and adequate treatment outcomes. For example, the type and/or style of the patient interface device used is an important component for insuring patient compliance and for insuring that the minimum effective therapeutic pressure is prescribed to the patient.

The flow of gas supplied to a patient encounters resistance associated with, for instance, the patient's nasal cavity and/or oral cavity. These resistances may vary from patient to patient. A patient suffering from a number of intranasal pathological conditions (such as and without limitation, deviated septum, polyps, hypertrophic turbinates and spurs, trauma, congenital problems, sinus infections, etc.) may have a high nasal resistance. If a nasal mask is the type selected for such a patient, the minimum effective therapeutic pressure that must be prescribed to overcome the patient's elevated nasal resistance may be relatively high. However, if a nasal/oral mask is the type selected, the minimum effective therapeutic pressure may be relatively low because the patient will also be able to breathe through his/her mouth, thus bypassing the elevated nasal resistance. Prescribing a lower minimum effective therapeutic pressure increases the likelihood that the patient will comply with his/her therapy.

### SUMMARY OF THE INVENTION

Accordingly, it is an object of the present invention to provide a method of selecting an optimum patient interface device for treating a patient suffering from sleep disordered breathing that overcomes the shortcomings of conventional techniques. This object is achieved according to one embodiment of the present invention by providing a system and/or method as described herein that allows the therapist or other caregiver to select a device from a range or group of interface devices with improved efficiency. In an exemplary embodiment, the potential range or group of interface devices from which the therapist or other caregiver selected may be greatly narrowed or reduced, thus decreasing the time required to select a mask while increasing the odds that a selected mask is best suited for a particular patient, hence effectively improving the overall selection process.

Embodiments of the invention generally utilize an initial questioning of the patient to gather information which is analyzed to determine the presence and/or level of one or more comorbidities and/or anxiety causing issues of a patient. The results of such questioning are then used to reduce the number of potential interface devices to be considered for the patient. Embodiments of the invention may identify a particular suggested optimum device or devices from within a larger grouping or, alternatively, may identify a particular unsuitable device or devices to be eliminated from consideration from within a larger grouping. By taking into consideration one or more comorbidities and/or anxiety causing issues of a patient when selecting a treatment device, the patient's adherence to prescribed therapy is positively affected.

In accordance with one aspect of the present invention, a method for reducing a potential group of patient interface devices adapted to treat disordered breathing of a patient to an optimized group is provided. The method comprises prescreening the patient, determining a measure of a comorbidity of the patient from the prescreening; comparing the measure of the comorbidity to a corresponding predetermined threshold; and determining the optimized group from the potential group.

The optimized group may consist of a particular device and the method may further comprise performing a titration with the particular device disposed on the patient.

The method may further comprise selecting a particular device from the optimized group and performing a titration with the particular device disposed on the patient.

Prescreening the patient may comprise providing a number of questions to the patient and determining a measure of a comorbidity of the patient may comprise receiving responses to at least some of the number of questions and analyzing the responses.

Determining a measure of the comorbidity of the patient may comprise determining a measure of one or more of the nasal stuffiness, claustrophobia, asthma or COPD of the patient.

At least one response of the received responses may comprise a numerical response within a predetermined numerical range and analyzing the responses may comprise tabulating the numerical responses.

Providing a number of questions to the patient may comprise providing a physical questionnaire to the patient.

Providing a number of questions to the patient may comprise providing a number of questions via a website.

The particular device may comprise one of a patient interface device or a mandibular advancement device. The patient interface device may comprise one of nasal prongs, a nasal mask, a full nasal/oral mask, or a total face mask.

The particular device may comprise one of nasal prongs, a nasal mask, a full nasal/oral mask, or a total face mask.

A number of the questions may relate to the anticipated anxiety of a patient in a number of scenarios.

The method may further comprise determining a measure of a second comorbidity of the patient from the prescreening, comparing the measure of the second comorbidity to a corresponding predetermined threshold, and determining the optimized group from the potential group.

Determining a measure of a comorbidity of the patient from the prescreening may comprise determining a measure of the nasal stuffiness of the patient and determining a measure of a second comorbidity of the patient from the prescreening may comprise determining a measure of the claustrophobia of the patient.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a schematic view of an example system for treating disordered breathing in a patient;

FIG. 2 illustrates an operational process for selecting a device adapted to treat disordered breathing according to one embodiment of the present invention;

FIG. 3 illustrates graphically an embodiment of the present invention in which two comorbidities of a patient are considered in narrowing a selection of potential interface devices for a patient; and

FIGS. 4A-4D illustrate non-limiting example patient interface devices which may be employed with embodiments of the present invention.

### DETAILED DESCRIPTION OF THE EXEMPLARY EMBODIMENTS

Directional phrases used herein, such as, for example, left, right, clockwise, counterclockwise, top, bottom, up, down, and derivatives thereof, relate to the orientation of the elements shown in the drawings and are not limiting upon the claims unless expressly recited therein.

As employed herein, the term “number” shall mean one or more than one and the singular form of “a”, “an”, and “the” include plural referents unless the context clearly indicates otherwise. As employed herein, the statement that two or more parts are “connected” or “coupled” together shall mean that the parts are joined together either directly or joined together through one or more intermediate parts. Further, as employed herein, the statement that two or more parts are “attached” shall mean that the parts are joined together directly.

As employed herein, the term “disordered breathing” shall be used to refer to breathing abnormalities which may occur while a patient is one or both of asleep or awake, examples of which may include, without limitation, increased upper airway resistance (including snoring), upper airway resistance syndrome (UARS), Cheyne-Stokes respiration (CSR), and obstructive sleep apnea-hypopnea syndrome (OSAHS) as well as any combination thereof. As employed herein, the term “patient” shall refer to the person requiring treatment for disordered breathing. As employed herein, the term “therapist” shall refer to the person or persons determining and providing the treatment or system for treating the patient.

As employed herein, the term “comorbidity” or “comorbidities” shall refer to other diseases or conditions which may afflict a patient to some degree in addition to disordered breathing. As employed herein, the term “nasal stuffiness” shall be used to refer to nasal congestion experienced by a patient which may result from the swelling of nasal tissues and blood vessels with excess fluid, causing a “stuffy” feeling. Example causes of nasal congestion include, without limitation, a cold, influenza and allergies. As employed herein, the term “claustrophobia” shall be used to refer to anxiety or abnormal dread of being in closed or narrow spaces and or anxiety caused by fear of suffocation experienced by a patient.

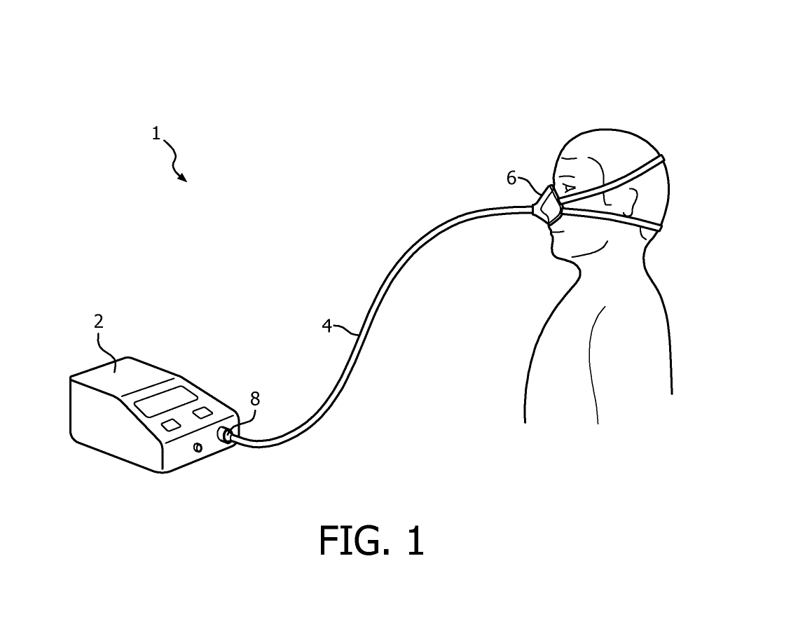

An example of a system 1 for use in treating disordered breathing is shown in FIG. 1. In system 1, treatment is provided by delivering a flow of breathing gas to a patient (not numbered). System 1 includes a flow generator 2, a patient circuit 4, and a patient interface device 6, as may be selected using the systems or methods described herein.

Flow generator 2 is structured to generate a flow of breathing gas which is communicated from an outlet 8 on flow generator 2, through patient circuit 4, through patient interface device 6, and to the airway of the patient. Although it is contemplated that other devices may be used, a REMstar® M Series therapy device available from Philips Respironics of Murrysville, Pa. is employed as the flow generator 2 in FIG. 1. Additionally, although a nasal mask is illustrated as being used in FIG. 1, it is contemplated that other types of patient interface devices (e.g., nasal prongs; full oral/nasal masks; total face masks; etc.) may be employed as patient interface device 6 without varying from the scope of the present invention.

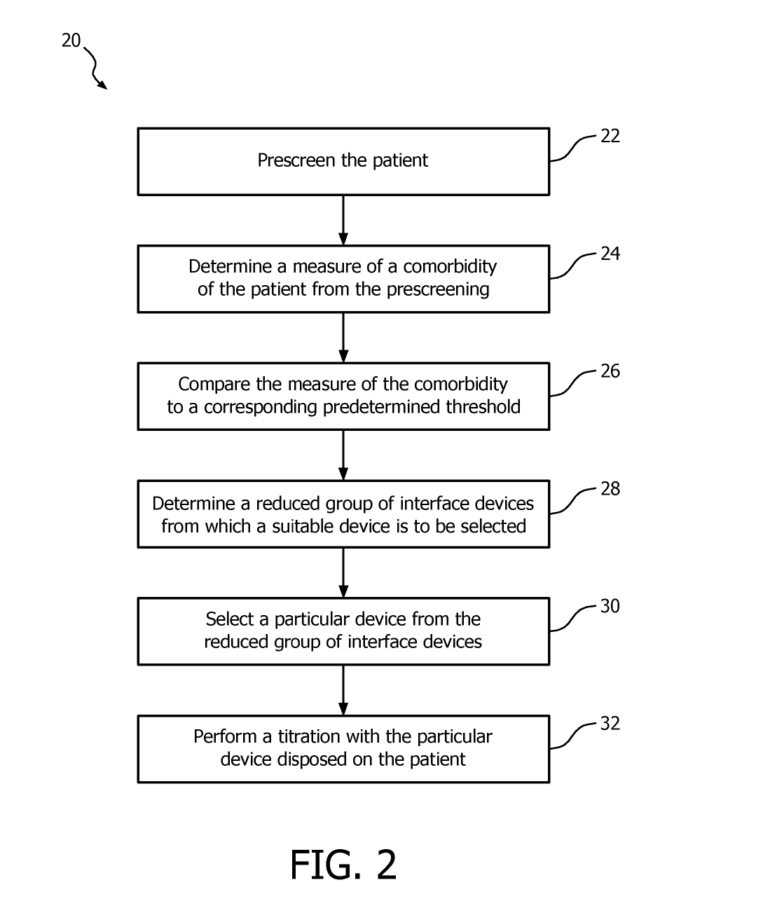

FIG. 2 illustrates an operational process 20 of a method in accordance with an example embodiment of the present invention for reducing a potential group of patient interface devices adapted to treat sleep disordered breathing of a patient to a lesser optimized group. Embodiments of the present invention may be used to assist in selecting from among potential interface devices such as, for example, without limitation, nasal prongs, nasal masks, full nasal/oral masks, total face masks, or mandibular advancement devices. It is to be appreciated that embodiments of the present invention may be applied to reduce initial groups of two or more interface devices to optimized groups of lesser quantities which may include one or more interface devices. By reducing the number of potential interface devices from which a therapist may choose using the system and/or methods described herein, the overall time spent selecting a device for a patient is reduced while the likelihood of selecting a device which appeals to the patient, and thus is worn/used as prescribed, is increased.

Referring to FIG. 2, process 20 begins with a prescreening of the patient for the presence and/or extent of one or more of a number of comorbidities in addition to disordered breathing, such as shown in operation 22. In an example embodiment of the present concept, prescreening of the patient is carried out by providing a plurality of questions to the patient, with a number of the questions being directed toward determining the presence/extent of one or more comorbidities and/or a measure of the patient's anxiety which results from the presence of such comorbidities. Comorbidities of potential concern may include, for example, without limitation, nasal resistance, claustrophobia, asthma or chronic obstructive pulmonary disease (COPD). It is to be appreciated that the more anxiety a given interface device causes a patient, the less likely the patient will adhere to a prescribed therapy regimen.

In an example embodiment of the present invention, a number of questions are provided to a patient in which the patient is generally prompted to “indicate the degree to which you would feel anxious in the following situation, experienced or imagined, by selecting the most appropriate number”. The patient is then given the option of answering one of: “0—Not at all anxious”; “1—Slightly anxious”; “2—Moderately anxious”; “3—Very anxious”; or “4—Extremely anxious”. Some example scenario questions employed to gauge the presence/extent of anxiety related to claustrophobia of the patient include (for example, without limitation): “having a bad cold and finding it difficult to breath”; “snorkeling in a safe practice tank for 15 minutes”; “wearing a dust mask on a hot day for 15 minutes”; and “wearing a hot full-face covering Halloween mask for 15 minutes”. Some example scenario question employed to gauge the presence/extent of a patient's nasal stuffiness include (for example, without limitation): “over the last 3 months how often, on average, did you have facial pain and/or pressure” and “over the last 3 months how often, on average, did you have a nasal obstruction (severe congestion/ blockage)”.

From such questions as exemplified above, a numerical measure of the comorbidity or comorbitities of the patient may be obtained. It is to be appreciated that such questions may be provided to the patient in various manners without varying from the scope of the present invention. For example, without limitation, such questions may be provided through a physical questionnaire which may be completed and returned to the therapist or provided through a website interface through which questions could be initially provided to the patient and subsequent responses submitted to the therapist. In example embodiments of the present invention, questions are provided which elicit responses which may be autoscored by a computer or other suitable processing means.

Continuing to refer to FIG. 2, at operation 24, a measure of a comorbidity or multiple comorbidities of the patient is determined from the results of the prescreening. In an example embodiment of the present invention, such determination is made by analyzing the responses received from the patient to at least some of the number of questions and determining the measure from such analysis. In the example embodiment discussed above in which potential responses ranged from “0—Not at all anxious” to “4—Extremely anxious”, analyzing the responses comprises tallying the numerical responses received to determine a numerical measure or score for the comorbidity of interest.

As shown in operation 26 of FIG. 2, once the measure of the comorbidity or comorbidities is/are determined, the measure of the comorbidity or comorbidities is/are compared to a corresponding threshold value or values which generally delimits a predetermined group of potential interface devices into two or more smaller reduced groups of interface devices for treating disordered breathing. From such comparison(s), a reduced group of potential interface devices is determined, as shown in operation 28 of FIG. 2.

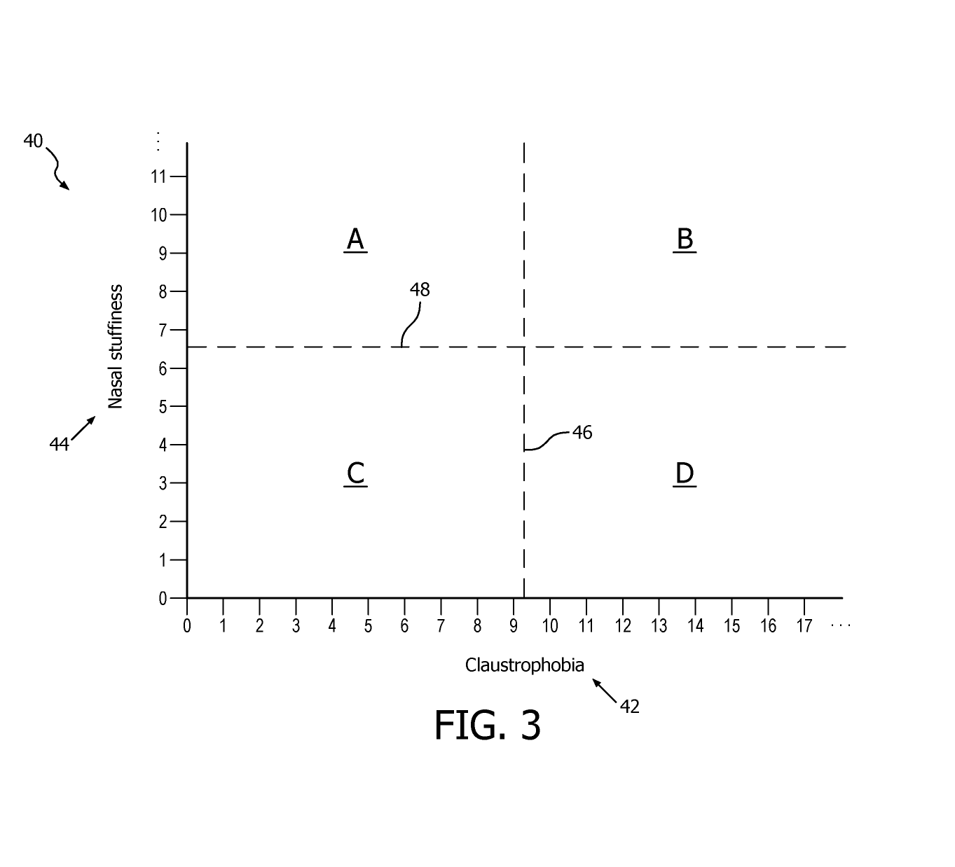

FIG. 3 illustrates a graphical representation 40 of an example embodiment of the present invention in which two comorbidities were employed in reducing a group of potential interface devices into a smaller reduced group. More particularly, measured values of the comorbidities of claustrophobia 42 (x-axis) and nasal stuffiness 44 (y-axis) were considered in reducing a potential group of patient interface devices down to one of four smaller, optimized groups, as denoted generally by the quadrants A, B, C and D delimited by the claustrophobia threshold 46 and the nasal stuffiness threshold 48.

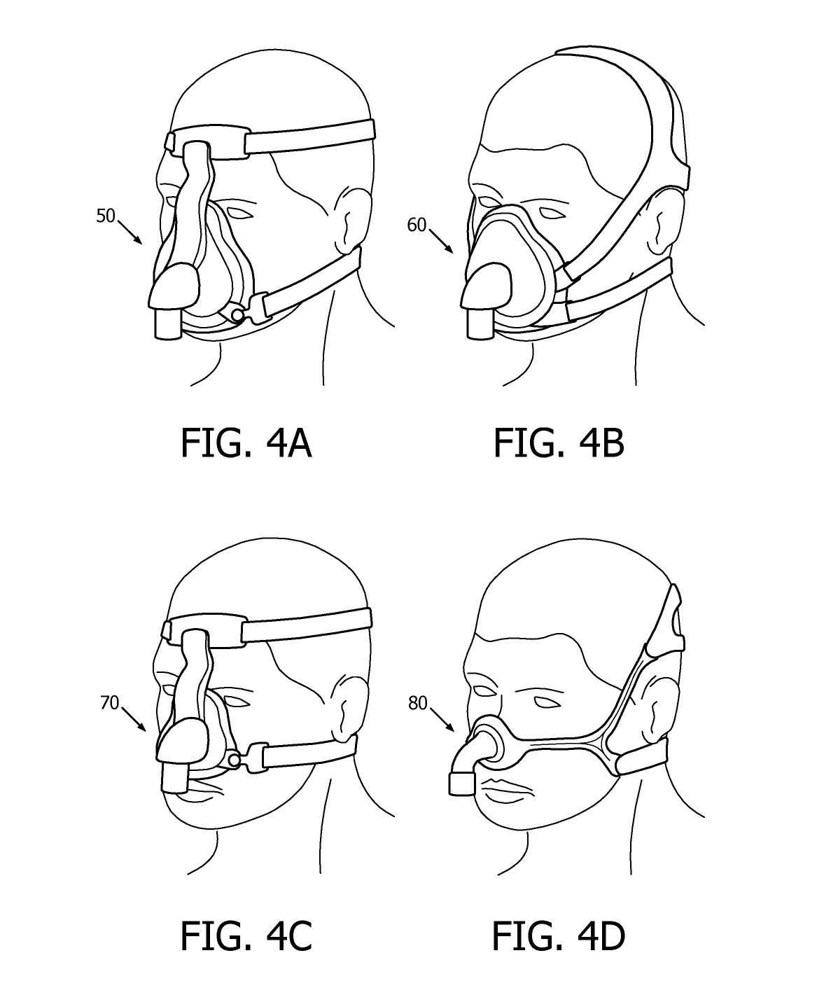

For example, a patient having a high nasal stuffiness measure and a low claustrophobia measure would likely benefit from a device categorized in quadrant A, such as a full face mask with forehead support such as, for example, without limitation a ComfortGel™ full mask manufactured and distributed by Philips Respironics of Murrysville, Pa., an example 50 of which is shown in FIG. 4A. Such mask is likely a good choice for a patient exhibiting such comorbidities as being a full face mask it is adapted to provide a flow of treatment gas to a patient suffering from high nasal stuffiness. Also, as the patient is generally not claustrophobic, the additional forehead support (not numbered) is likely to not be a deterrence to the patient wearing the device.

An another example, a patient having a high nasal stuffiness measure and a high claustrophobia measure would likely benefit from a device categorized in quadrant B, such as a full face mask without a forehead support such as, for example, without limitation a FullLife™ full face mask manufactured and distributed by Philips Respironics of Murrysville, Pa., an example 60 of which is shown in FIG. 4B. Such mask is likely a good choice for a patient exhibiting such comorbidities as being a full face mask it is adapted to provide a flow of treatment gas to a patient suffering from high nasal stuffiness. Also, as the patient is claustrophobic, the general openness of the mask 60 due to lack of a forehead support is likely to be appealing to the patient.

As yet another example, a patient having a low nasal stuffiness measure and a low claustrophobia measure would likely benefit from a device categorized in quadrant C, such as a nasal mask having a forehead support such as, for example, without limitation, a ComfortGel Blue™ nasal mask manufactured and distributed by Philips Respironics of Murrysville, Pa., an example 70 of which is shown in FIG. 4C. Such mask is likely a good choice for a patient exhibiting such comorbidities as being a nasal mask it is suitable to provide a flow of treatment gas to a patient suffering from low nasal stuffiness. Also, as the patient is generally not claustrophobic, the additional forehead support (not numbered) is likely to not be a deterrence to the patient wearing the device.

As a further example, a patient having a low nasal stuffiness measure and a high claustrophobia measure would likely benefit from a device categorized in quadrant D, such as a nasal mask without a forehead support such as, for example, without limitation, a Wisp™ nasal mask manufactured and distributed by Philips Respironics of Murrysville, Pa., an example 80 of which is shown in FIG. 4D. Such mask is likely a good choice for a patient exhibiting such comorbidities as being a nasal mask it is suitable to provide a flow of treatment gas to a patient suffering from low nasal stuffiness. Also, as the patient is claustrophobic, the general openness of the mask 80 due to lack of a forehead support is likely to be appealing to the patient.

From the foregoing example, it is to be appreciated that from an initial potential group of four devices, an optimum single device was determined using values of both of the comorbidities of claustrophobia and nasal stuffiness. In such example, a single comorbidity could be employed to narrow the initial group of four down to two potential candidates.

Although particular values are shown for nasal stuffiness and claustrophobia measures are shown in FIG. 3, it is to be appreciated that such values are shown only for example purposes and are not intended to be limiting upon embodiments of the present invention as such values or range thereof may vary depending on one or more of the type and quantity of survey questions asked of a patient. Also, it is to be appreciated that the placement of the thresholds 46 and 48 is also provided for example purposes only and that the particular placement of such thresholds may vary depending on one or more of the type and quantity of survey questions asked of a patient as well as the beginning group of potential interface devices being narrowed.

Referring again to FIG. 2, after the reduced group of interface devices is determined in operation 28, a particular device for treating the patient is selected from the reduced group, as shown in operation 30 of FIG. 2, and subsequently, the particular device is fitted to the patient (such as shown, for example, without limitation, in FIG. 1) and a titration is performed, as shown in operation 32 of FIG. 2, to ensure suitability of the device for the patient.

It can be appreciated that the present invention provide a method in which one or more comorbidities of the patient (e.g., without limitation, nasal resistance, claustrophobia, asthma, chronic obstructive pulmonary disease (COPD)) and/or the effects on breathing caused by these comorbidities, are considered during the selection of an appropriate device for treating disordered breathing.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For instance, although discussed in context of selecting a desirable patient interface for delivery of a flow of breathing gas, it is contemplated that operational process 30 can easily be adapted for selecting other devices used to treat disordered breathing. For example, operational process 30 may be adapted such that an oral appliance, such as mandibular advancement device (MAD), is selected when dictated by the measured comorbidity or comorbidities. For example, without limitation, in a situation where a patient exhibits a high measure of claustrophobia and a low level of nasal stuffiness, a mandibular advancement device may be selected.

It is to be appreciated that the operations and methods described herein may be readily encoded, in whole or in-part, on machine readable storage medium(s) which may be readily employed by a processing device or devices to automatically carry out all or portions of the methods described herein. For example, in an embodiment of the present invention, a patient is presented with questions on a screen related to a computing device (e.g., without limitation, a desktop computer, smart phone, etc.). The patient then inputs (e.g., via keyboard, mouse, touchscreen, etc.) responses to the questions into the device. The device then analyzes the responses received from the patient and then employ the operations described herein to output a suggested device or devices either directly to the patient or a therapist.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A method for reducing a potential group of patient interface devices adapted to treat disordered breathing of a patient to an optimized group, the method comprising:
prescreening the patient;
determining a measure of a comorbidity of the patient from the prescreening;
comparing the measure of the comorbidity to a corresponding predetermined threshold; and
determining the optimized group from the potential group based at least inpart on the measure of the comorbidity.

2. The method of claim 1, wherein the optimized group consists of a particular device and wherein the method further comprises performing a titration with the particular device disposed on the patient.

3. The method of claim 1, further comprising selecting a particular device from the optimized group and performing a titration with the particular device disposed on the patient.

4. The method of claim 1, wherein prescreening the patient comprises providing a number of questions to the patient and wherein determining a measure of a comorbidity of the patient comprises receiving responses to at least some of the number of questions and analyzing the responses.

5. The method of claim 1, wherein determining a measure of the comorbidity of the patient comprises determining a measure of one or more of the nasal stuffiness, claustrophobia, asthma or COPD of the patient.

6. The method of claim 4, wherein at least one response of the received responses comprises a numerical response within a predetermined numerical range.

7. The method of claim 6, wherein analyzing the responses comprises tabulating the numerical responses.

8. The method of claim 4, wherein providing a number of questions to the patient comprises providing a physical questionnaire to the patient.

9. The method of claim 4, wherein providing a number of questions to the patient comprises providing a number of questions via a website.

10. The method of claim 2, wherein the particular device comprises one of a patient interface device or a mandibular advancement device.

11. The method of claim 10, the patient interface device comprises one of nasal prongs, a nasal mask, a full nasal/oral mask, or a total face mask.

12. The method of claim 3, wherein the particular device comprises one nasal prongs, a nasal mask, a full nasal/oral mask, or a total face mask.

13. The method of claim 4, wherein a number of the questions relate to the anticipated anxiety of a patient in a number of scenarios.

14. The method of claim 1, further comprising:
determining a measure of a second comorbidity of the patient from the prescreening;
comparing the measure of the second comorbidity to a corresponding predetermined threshold; and
determining the optimized group from the potential group.

15. The method of claim 14, wherein determining a measure of a comorbidity of the patient from the prescreening comprises determining a measure of the nasal stuffiness of the patient and wherein determining a measure of a second comorbidity of the patient from the prescreening comprises determining a measure of the claustrophobia of the patient.
